(19) United States
(12) Patent Application Publication
(10) Pub. No.: US 2018/0318124 A1
(43) Pub. Date: Nov. 8, 2018

(54) BRACES FOR SPINAL DEFORMITIES AND METHODS OF USE

(71) Applicant: CHINE, LLC, Exeter, NH (US)

(72) Inventors: John MACMAHON, Exeter, NH (US); Luke STIKELEATHER, Oakton, VA (US); Tom SAUL, Moss Beach, CA (US)

(21) Appl. No.: 15/773,425

(22) PCT Filed: Nov. 4, 2016
(86) PCT No.: PCT/US16/60658

§ 371 Date: May 3, 2018

Related U.S. Application Data
(60) Provisional application No. 62/250,958, filed on Nov. 4, 2015.

(51) Int. Cl. A61F 5/02; A61B 6/00

(52) U.S. Cl. CPC A61F 5/024; A61F 2005/0188; A61B 6/505

(57) ABSTRACT

Wearable devices for spinal deformities, including a wearable brace configured to be worn about a patient's torso, at least one inflation bladder carried directly or indirectly by an inner surface of the brace, and positioned such that inflation of the bladder with a fluid applies a pressure to generate a force on the patient at a targeted location, at least one force sensor carried directly or indirectly by a surface of the brace, to measure the force independent of the bladder, and the force sensor adapted to output a signal indicative of a force applied on the sensor.

### CROSS REFERENCE TO RELATED APPLICATIONS

This application claims the priority of U.S. Provisional Application No. 62/250,958, filed Nov. 4, 2015, which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference. This application incorporates by reference herein the disclosure of U.S. Pub. No. 2015/0257915, published Sep. 17, 2015.

### BACKGROUND

Improved diagnostic and therapeutic braces, and methods of using them, are needed for individuals with spinal deformities such as scoliosis.

### SUMMARY OF THE DISCLOSURE

One aspect of the disclosure is a wearable device for spinal deformities, comprising: a wearable brace configured to be worn about a patient's torso; at least one inflation bladder carried directly or indirectly by an inner surface of the brace, and positioned such that inflation of the bladder with a fluid applies a pressure to generate a force on the patient at a targeted location, optionally to treat scoliosis; and at least one force sensor carried directly or indirectly by a surface of the brace, to measure the force independent of the bladder, and the force sensor adapted to output a signal indicative of a force applied on the sensor.

In some embodiments, the force sensor is carried directly or indirectly by the inner surface of the brace.

In some embodiments, resistance of the sensor changes in response to a force applied to the force sensor.

In some embodiments, the force sensor is positioned to sense a force applied by the inflation bladder on the patient.

In some embodiments, the force sensor and the bladder are co-located between the patient and the brace.

In some embodiments, the device further comprises a pressure sensor adapted to sense pressure within the bladder.

One aspect of the disclosure is a system for spinal deformities, comprising: a wearable brace configured to be worn about a person's torso; at least one inflation bladder carried directly or indirectly by an inner surface of the brace, and positioned such that inflation of the bladder with a fluid applies a pressure to generate a force on the patient at a targeted location to treat scoliosis; and at least one force sensor carried directly or indirectly by a surface of the brace, to measure the force independent of the bladder, and the force sensor adapted to output a signal indicative of a force applied on the sensor; and a signal conditioning module with a communication element to communicate force sensor outputs to an external device.

In some embodiments, the system further comprises a pressure sensor adapted to sense pressure within the bladder, and can optionally communicate pressure sensor outputs from the pressure sensor to the external device. The external device can be adapted to keep a time history of at least one of force and pressure.

In some embodiments, the force sensor is carried directly or indirectly by the inner surface of the brace.

In some embodiments, the force sensor is positioned to sense a force applied by the inflation bladder on the patient.

In some embodiments, the signal conditioning module is housed in a housing coupled directly or indirectly to an outer surface of the brace.

In some embodiments, the force sensor and the bladder are co-located between the patient and the brace.

One aspect of the disclosure is a method for spinal deformities, optionally diagnostic or therapeutic, comprising: positioning a brace about a patient's torso, the brace including at least one inflatable bladder carried directly or indirectly by an inner surface of the brace, and at least one of a force sensor and a pressure sensor carried directly or indirectly by the brace, to measure the force and/or pressure independent of the bladder, and the force sensor and/or pressure sensor adapted to output a signal indicative of a force or pressure applied on the sensor; and delivering a fluid, such as air, to the bladder to inflate the bladder, which applies a pressure to generate a force on the patient at a targeted location, optionally to treat scoliosis if used as a therapeutic brace.

In some embodiments, the method includes sensing pressure in the bladder, and optionally storing the sensed pressure, optionally in an external device.

### BRIEF DESCRIPTION OF THE DRAWINGS

FIG. 1 illustrates an exemplary fixed diagnostic standing frame, which can be used to create a force corrected image of a patient.

FIG. 2 illustrates an exemplary fixed diagnostic sitting frame, which can be used to create a force corrected image of a patient.

FIG. 3 illustrates an exemplary wearable brace.

FIG. 4 illustrates an exemplary wearable brace.

FIGS. 5A and 5B illustrate an exemplary wearable brace.

FIG. 6 illustrates an exemplary wearable brace.

FIG. 7 illustrates an exemplary wearable brace with integrated sensor.

FIG. 8 illustrates an exemplary wearable brace with integrated sensor.

FIG. 9 illustrates an exemplary wearable brace with integrated sensor.

FIG. 10 illustrates an exemplary wearable brace with integrated sensor.

FIG. 11 illustrates an exemplary wearable brace with integrated sensor.

FIG. 12 illustrates an exemplary system including a wearable brace and an external device.

FIG. 13 illustrates an exemplary wearable monitoring system in which sensors are integrated into a sensing garment.

### DETAILED DESCRIPTION OF THE DISCLOSURE

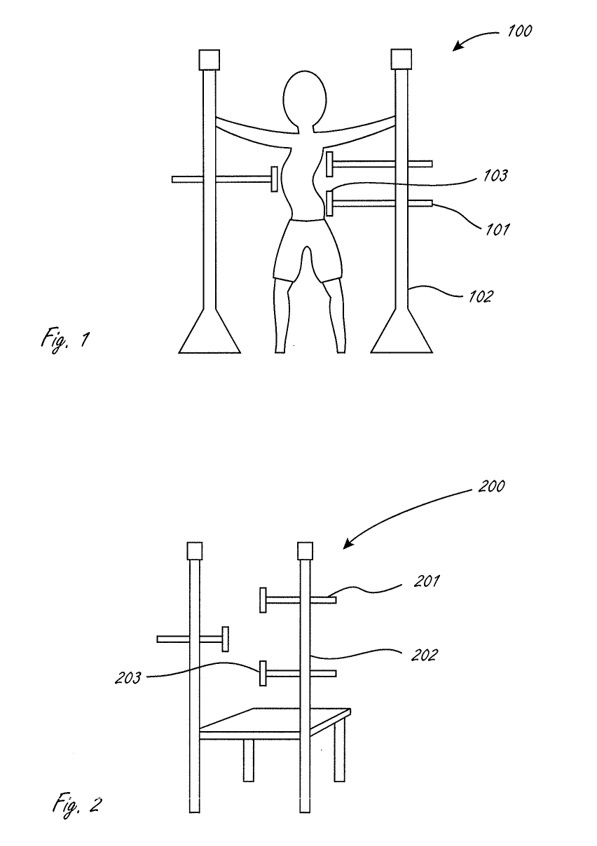

FIG. 1 illustrates a fixed diagnostic standing frame 100 adapted and configured for use in characterizing the relationships between the magnitude of a distension, the corresponding change in Cobb angle, and the resultant interface pressure associated with the application of such a distension. Such a system has an exemplary use in an x-ray or other imaging system capable of imaging the position and displacements of the spine as a function of displacement of at least one of the plurality of distenders 101. The system as illustrated comprises three distenders 101, although more of fewer may be used. The distenders in some embodiments additionally comprise a means of monitoring displacements particularly in the plane including by the two frame elements 102. The distenders are interfaced with a frame structure or element 102. Each distender additionally comprises a distender interface 103, which will typically be comprised of an element which is adapted to conform to a body surface to which it will interface when in use. Such surfaces, although not limited to, may comprise any selection or combination of foams, fluid or air filled bladders, elements fabricated to conform to the contour of the body being evaluated. Each distender interface may additionally comprise any selection or combination of an interface surface providing contrast in the generated image, pressure sensors, load sensors. Pressure and load sensors allow for the characterization of the pressures generated by the distenders on the skin of the patient's body.

In an exemplary procedure using the system of FIG. 1, a patient is positioned, such as by standing, between the frame elements 102. The distender elements 101 will then be adjusted so that the distender interfaces 103 interface with appropriate portions of the body as illustrated such that no or minimal pressure is applied by the distender elements 103. An image will then be acquired using an imaging device, such as an x-ray machine. Next, distender elements 101 will be adjusted such that a maximum of 2 psi is applied on the interface surface of any distender interface 103, and an image is then acquired. The 2 psi limitation will minimize the likelihood of the patient developing ulcers. The order of the method steps can be modified in some procedures, and not every step necessarily needs to be carried out in the procedures.

In alternate procedures, more images may be acquired at more than two distensions and relationships generated comprising the relationship between measured Cobb angle, distensions, and interface pressures. For the purposes of such diagnostic activities more than 2 psi may be applied for short periods of time. Such measurements are useful in determining a contour for a therapeutic brace, which limits pressures to less than 2 psi for some portion of time while applying a maximum distension during normal activities. Such measurements additionally provide information on the effectivity of a given distension as time goes on when using a therapeutic brace as described herein.

When the conformal element 103 comprises an element fabricated to conform to the contour of the body being evaluated, a convenient means of fabricating the element is via 3D printing.

In some embodiments one or more distender interfaces 103 may be hydraulic. In the process of brace making today, the brace maker may scan the patient in the standing position. This gives them an uncorrected version of the patient's body in 3D format. Then the brace maker must rely on x-rays and previous experience to modify that accurate model to a proposed “Virtual Corrected” brace. This is a very subjective step. The resulting brace is a best effort of the experience of the brace maker at approximating the final brace geometry from a standing patient scan.

This disclosure, however, includes applying corrective forces to the patient with one or more inflation bladders while they are standing. The brace maker can then perform a corrected scan (image acquisition) with the starting point in the 3D modeling to be a “Force Corrected” brace, which is the fundamental objective of the brace maker. What is happening then is that instead of having to apply subjective years of experience to approximate what forces and geometry it takes to correct the patient, the embodiments herein (e.g., FIG. 1) would provide the actual forces applied and the actual geometry of the “Corrected Patient,” and then the imaging scan would occur, eliminating time and improving fit of the brace, increasing clinical impact.

Additionally, the apparatus, by sensing force and/or pressure, allows us to know the forces necessary to get into that position to optimize fit and function, as a brace too tight would not be worn. Feedback during fitting in the apparatus would result in greater confidence of the fit, form and clinical benefit, while reducing a subjective step.

This “Force Corrected” scan (imaging) results in an objectively acquired 3D imaged model, or in some cases a 2D x-ray, that would allow a beginner brace maker to get experienced brace maker models. Integrating an imaging system into the design would further accelerate and streamline the results and adoption of the system. Known imaging modality systems can be used.

The novel benefits of recording and correcting with both force and pressure can be in that forces correct the geometry, but pressure on the patient determines the ergonomic comfort. Combining these two is particularly powerful to get the most geometric benefit with ergonomics that would provide comfort. Since patients may be prescribed to wear the brace for up to 20 hours fit is very relevant.

The diagnostic frames herein can apply forces to correct a spinal deformity while allowing subsequent imaging to produce “force Corrected” scan to be performed, imaging may be 2D or 3D.

The diagnostic frames herein can apply pressures to correct a spinal deformity while allowing subsequent imaging to produce “pressured Corrected” scan to be performed, imaging may be 2D or 3D.

The diagnostic frames herein can apply forces and pressures to correct a spinal deformity while allowing subsequent imaging to produce “Force and Pressure Corrected” scan to be performed, imaging may be 2D or 3D.

The diagnostic frames herein can include geometric bounds to support the force and/or pressure correcting members as well as in integrated scanning system to produce the subsequent imaging.

The diagnostic frames herein can include geometric bounds to support the force and pressure correcting members may be in communication with a remote sensors and controller to allow imaging to be performed either remotely by a center of excellence to a remote center or to be performed in an x-ray venue.

Any of the distenders in the diagnostic frames herein can include any combination of bladders and sensors herein, to provide any of the feedback information described herein.

FIG. 2 illustrates an alternative to the standing frame of FIG. 1, and configured to allow the patient to sit during imaging. The sitting frame 200 comprises a fixed diagnostic sitting frame 202. Interfaced with the sitting frame 202 are distenders 201, as illustrated 3 distenders are used. In other alternatives more or less distenders may be used. Each distender may comprise any combinations of displacement sensor, pressure sensor, load sensor. In some embodiments some or all distenders may comprise no sensing elements. Each distender element comprises a distender interface 203 which may additionally comprise one or more pressure sensors and/or one or more load sensors. In an alternate to the embodiment illustrated, the two distender interfaces 203 on the right may be attached to a single distender unit. In some embodiments the distenders 201 may be adjusted by hand, in others they may be comprised of electrically or hydraulically driven linear activators.

Any of the distenders and distender interfaces described herein may be used in any of the systems herein, such as systems 100 or 200.

In an alternate embodiment to that of FIG. 1 the frame 102 may be configured for use on a reclining patient. In such an embodiment the components comprising the system may additionally be fabricated for use in an MRI.

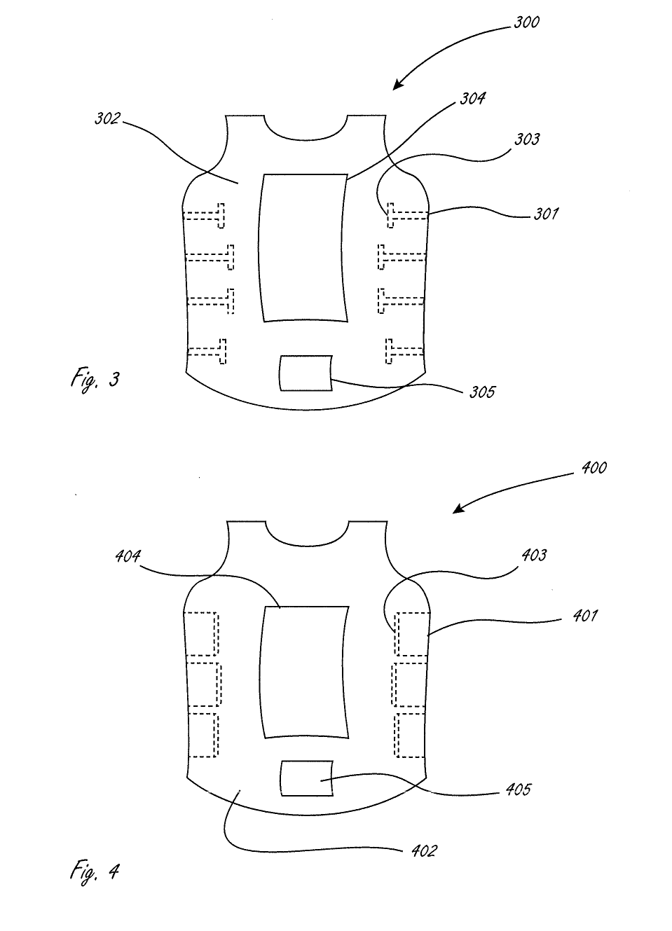

FIG. 3 illustrates a portable and wearable diagnostic brace 300 configure and adapted for use in an imaging system. The portable diagnostic brace 300 comprises a brace structure 302. The brace structure 302 is fabricated of a stiff material capable of supporting the loads generated by the plurality of distenders 301. As illustrated, system 300 comprises 8 distenders 301, but other numbers may be used as described herein with fixed systems, such as at least one distender. Each of the distenders 301 may comprise no or any combinations of displacement sensor, pressure sensor, and load sensor, any of which may be co-located with any other. Each of the distenders 301 comprises a distender interface 303 which in turn may comprise features as described for other distender interfaces described herein. The brace 302 additionally comprises an optional window 304 to allow for x-ray or other imaging modality lucency. The portable diagnostic brace 300 in addition may comprise an optional RF interface 305 configured to transmit information from the sensors comprised in the system to a remote recording and/or processing device not shown.

As illustrated, the distenders are disposed on the inside of the brace, but in alternate embodiments they may be disposed on the outside of the brace, or in some embodiments may be disposed partially inside and partially outside the brace.

FIG. 4 illustrates portable diagnostic brace 400 similar to that of system 300. In the system of FIG. 4 the at least one distender 401 (six shown in this example) include at least one hydraulic or pneumatic bladders, and the distender interfaces 403 are the interface surface of the bladder, all included on brace structure 402. The at least one distender is in this embodiment directly or indirectly carried by an inner surface of the brace body 402. Each distender 401 may comprise a pressure sensor and/or a displacement sensor for monitoring the pressure on the distender interface 403 and/or changes in position of the distender interface respectively. The one or more distenders 401 may also, or alternatively to the pressure sensors, include one or more force sensors. A force sensor can be co-located with a bladder and between the patient and the brace body, wherein the sensor is adapted to sense force applied to the patient by the bladder. In some embodiments an ultrasonic transducer is disposed in the bladder, or otherwise associated with the bladder, to allow for the monitoring of displacement and/or position of the distender interface surface 403. In some embodiments bladders are inflated manually, such as by a syringe or hand or powered pump. In others, the inflations may be controlled by a manifold comprising controllable valves and sourced by a pump. Inflation systems have not been illustrated. The brace 400 also comprises an imaging system lucent window 404 and an optional RF interface 405 capable of transmitting information from the sensors comprised in the system to a remote recording and/or processing device also not shown.

In alternate embodiments to those of FIGS. 3 and 4, the window 404 may be filled or replaced by an ultrasound sensing system capable of monitoring the conformation of the spine sufficiently to calculate a Cobb angle.

The diagnostic braces set forth herein can also be used as therapeutic braces in the treatment of scoliosis. For example, braces in FIGS. 3 and 4 can be used as therapeutic braces as well, wherein force is applied by the one or more bladders at targeted locations on the patient in the treatment of scoliosis. The sensors can provide feedback during the treatment.

An exemplary advantage of the portable systems is the capability to monitor the variation and magnitude of the interface pressure as a function of activities of daily living. Such measurements may be used to define an optimal shape for a therapeutic brace for daily use.

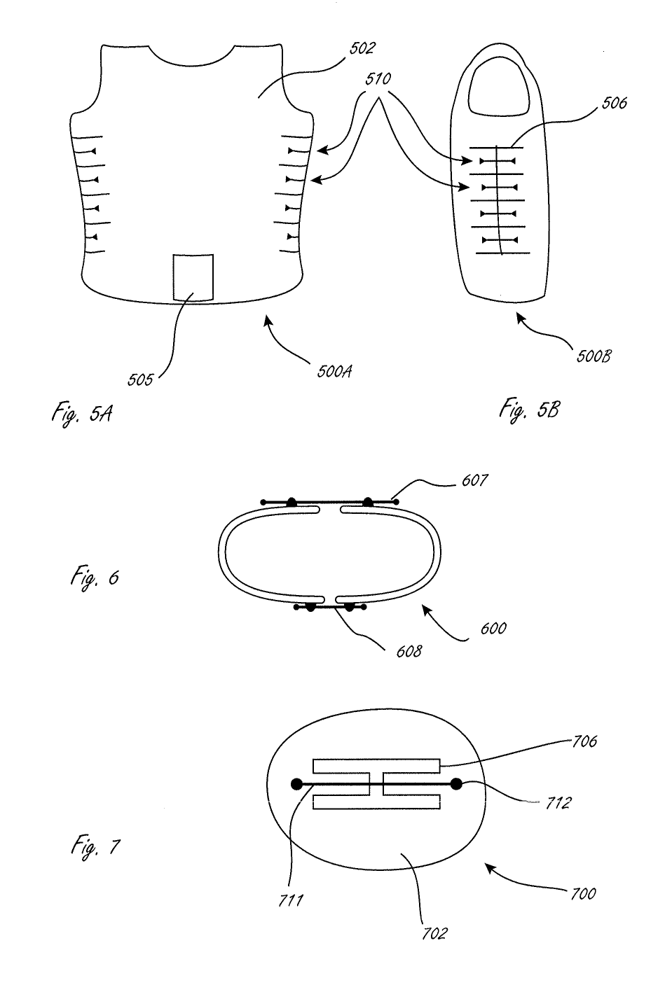

FIGS. 5A and 5B illustrate a therapeutic brace system 500 for daily use. The brace system 500 comprises at least one integral pressure sensor carried directly or indirectly by the brace body 502 (body may also be referred to herein as structure) for monitoring an interface pressure, which should be less than 2 psi on average for reasons as indicated herein. The integral pressure sensors 510 of brace 500 comprise sections of a slit 506 in the brace structure 502 spanned by a strain element or tension monitor 510. In some embodiments the strain element 510 is adjustable allowing for adjustment of a confirmation of the surface of the brace structure 502. The brace 500 comprises a signal conditioning module 505 with optional RF communications. In some embodiments the optional RF module is configured for communications to a cell phone to allow monitoring of interface pressures.

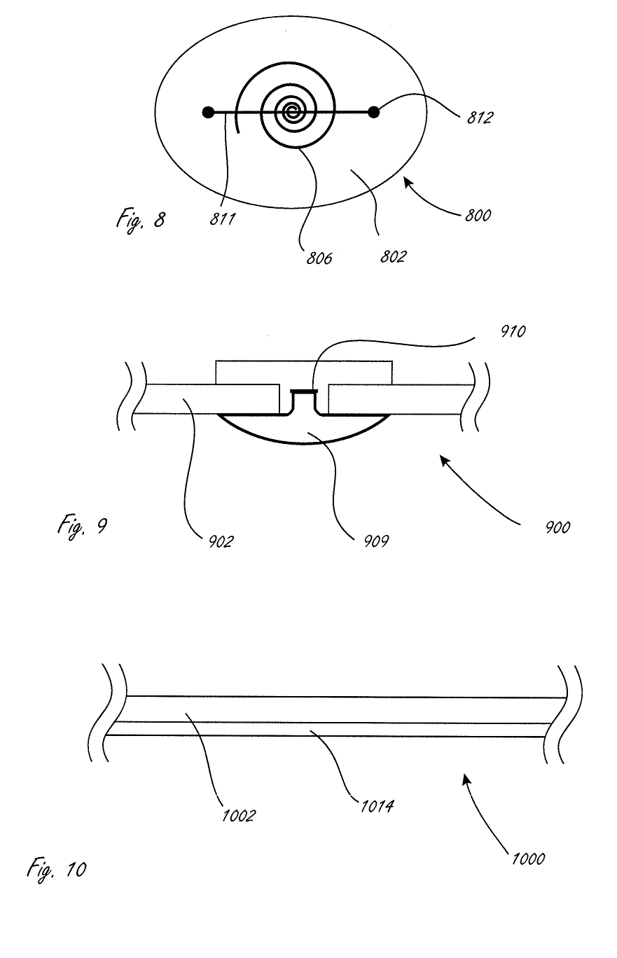

FIG. 6 illustrates another embodiment of a therapeutic brace 600. The brace 600 comprises tension monitors either in the closure 607 or closures and or in a seam opposite the closure 608. When the tension monitor is incorporated on the seam, multiple sensors may be incorporated. In an alternate embodiment the seam may run along the edges of the brace 600. FIGS. 7 thru 12 illustrate a number of possible embodiments for pressure sensors capable of integration into the structure of a therapeutic brace. Such structures should require minimal or no additional thickness in the brace structure.

FIG. 7 illustrates a therapeutic brace integral pressure sensor 700 similar to that illustrated in FIG. 5. Integral pressure sensor 700 comprises a slit 706 in the brace structure 702. A portion of the slit is spanned by a strain wire 711, which is affixed to two strain wire anchors 712 integrated into the brace structure.

FIG. 8 illustrates an alternate therapeutic brace integral pressure sensor 800. In sensor 800 the slit 806 in brace structure 802 has a spiral shape. The strain wire 811 spanning the slit 806 and associated anchors 812 are similar to those shown in FIG. 7.

FIG. 9 illustrates another alternate therapeutic brace integral pressure sensor 900 integrated into the brace structure 902. The structure comprises a pressure gauge or force gauge 910 integrated with a fluid or solid interface 909 respectively.

FIG. 10 illustrates yet another embodiment of a therapeutic brace integral pressure sensor 1000. In the sensor 1000 the inner surface or portions of the surface of the brace 1002 are covered with a pressure film 1014.

FIG. 11 illustrates yet another embodiment of a therapeutic brace integral pressure sensor 1100 comprising sensor 1113 embedded in a slit in the brace structure 1102. In such embodiments the sensing material of sensor 1113 may comprise a conductive elastomeric material whose resistance across a given path will change as a result of deformations associated with surface pressure on the brace local to the sensor. Alternatively, the material may be a dielectric with conductive surfaces, and the sensor used in a capacitive mode.

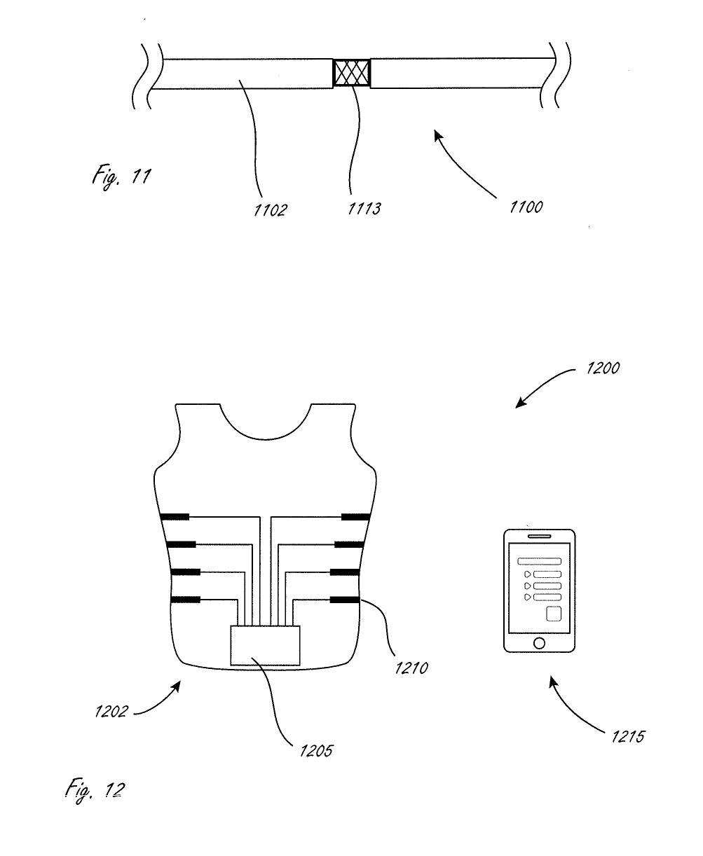

FIG. 12 illustrates a therapeutic brace system 1200 comprising any or any combination of the sensors described above. The system comprises brace 1202, which comprises a number of integrated pressure sensors 1210 carried directly or indirectly by a surface of the brace body. The pressure sensors 1210 are in communications with a signal conditioning module with an optional RF communications feature 1205 on the brace structure and a brace monitoring device 1215. As illustrated the brace monitoring device can be a smartphone or comparable device running an application to periodically record the sensor outputs.

Brace 1202 can be modified to include one or more bladders like in FIG. 4, which can be carried directly or indirectly by the inner surface of the brace body. Brace 1202 can include one or more force sensors (which may replace the pressure sensors), each of which can be co-located with an inflatable bladder, the force sensor adapted to generate an output that is indicative of the force applied to the patient by the bladder.

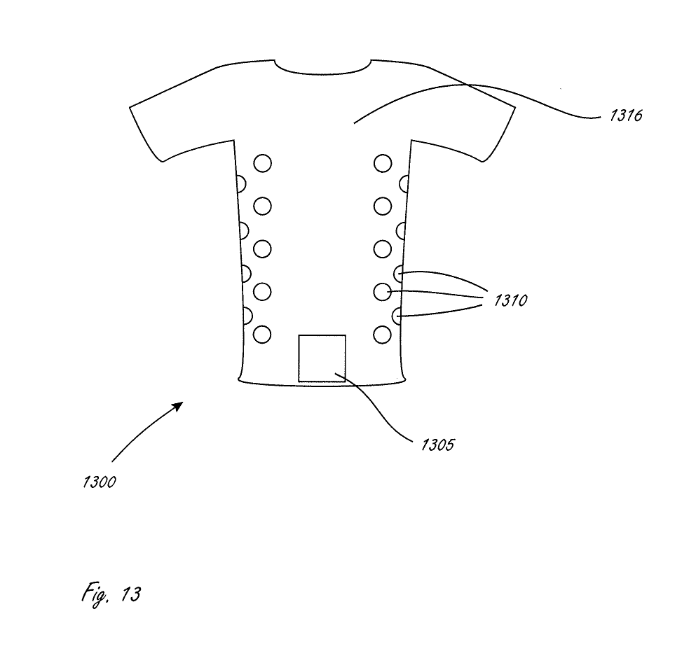

FIG. 13 illustrates an embodiment for a brace pressure monitoring system in which the pressure sensors are integrated into a pressure sensing garment 1300. The garment comprises a base garment 1316 comprising a set of pressure sensitive points 1310. Such a garment may comprise a material which provides some contrast in an imaging modality to allow for assessment of where the garment is in the image. The garment comprises a signal conditioning module with optional RF communications 1305.

Any of the braces herein that include one or more pressure sensors can also include one or more force sensors, which can be co-located with an inflation bladder to be able to sense force applied by the bladder to the patient. The inflation bladder and force sensor can be carried directly or indirectly by an inner surface of the brace body. Braces herein are configured to be worn about a patient's torso.

What is claimed is:

1. A method of obtaining a force or pressure corrected image of a patient, comprising:
positioning a patient with a spinal deformity within a standing or sitting frame, the standing or sitting frame comprising one or more distenders, at least one of the or more distenders comprising a bladder;
deforming the patient's spine by contacting the at least one bladder with the patient;
after the deforming step, measuring at least one of force on the patient and pressure within the at least one bladder; and
measuring the deformation of the spine.

2. The method of claim 1, wherein deforming the patient's spine comprises at least one of moving the one or more distenders toward the patient and inflating the at least one bladder.

3. The method of claim 2, wherein deforming comprises inflating the at least one bladder without moving the one or more distenders.

4. The method of claim 2, wherein deforming comprises moving the one or more distenders with an already inflated bladder into contact with the patient.

5. The method of claim 1, wherein measuring the deformation comprises imaging the patient after deforming the patient's spine.

6. The method of claim 1, wherein measuring the deformation comprises measuring displacements of the one or more distenders.

7. The method of claim 1, further comprising creating a brace body based on the measured information.

8. A standing or sitting frame for generating a force or pressure corrected image, comprising:
a standing frame comprising one or more distenders, at least one of the distenders comprising an inflation bladder disposed in an end region of the distende, the at least one bladder positioned to contact a patient standing or sitting within the frame, and at least one of a force sensor and a pressure sensor adapted to sense force applied to the patient by the bladder and/or pressure within the bladder.

9. A wearable device for spinal deformities, comprising:
a wearable brace configured to be worn about a patient's torso;
at least one inflation bladder carried directly or indirectly by an inner surface of the brace, and positioned such that inflation of the bladder with a fluid applies a pressure to generate a force on the patient at a targeted location to treat scoliosis; and
at least one force sensor carried directly or indirectly by a surface of the brace, to measure the force independent of the bladder, and the force sensor adapted to output a signal indicative of a force applied on the sensor.

10. The device of claim 9, wherein the force sensor is carried directly or indirectly by the inner surface of the brace.

11. The device of claim 9, wherein resistance of the sensor changes in response to a force applied to the force sensor.

12. The device of claim 9, wherein the force sensor is positioned to sense a force applied by the inflation bladder on the patient.

13. The device of claim 9, wherein the force sensor and the bladder are co-located between the patient and the brace.

14. The device of claim 9, further comprising a pressure sensor adapted to sense pressure within the bladder.

15. A wearable device for spinal deformities, comprising:
a wearable brace configured to be worn about a patient's torso;
at least one inflation bladder carried directly or indirectly by an inner surface of the brace, and positioned such that inflation of the bladder with a fluid applies a pressure to generate a force on the patient at a targeted location; and
at least one force sensor carried directly or indirectly by a surface of the brace, to measure the force independent of the bladder, and the force sensor adapted to output a signal indicative of a force applied on the sensor.

16. The device of claim 15, wherein the force sensor is carried directly or indirectly by the inner surface of the brace.

17. The device of claim 15, wherein resistance of the sensor changes in response to a force applied to the force sensor.

18. The device of claim 15, wherein the force sensor is positioned to sense a force applied by the inflation bladder on the patient.

19. The device of claim 15, wherein the force sensor and the bladder are co-located between the patient and the brace.

20. The device of claim 15, further comprising a pressure sensor adapted to sense pressure within the bladder.

21. A system for spinal deformities, comprising:
a wearable brace configured to be worn about a person's torso;
at least one inflation bladder carried directly or indirectly by an inner surface of the brace, and positioned such that inflation of the bladder with a fluid applies a pressure to generate a force on the patient at a targeted location to treat scoliosis; and
at least one force sensor carried directly or indirectly by a surface of the brace, to measure the force independent of the bladder, and the force sensor adapted to output a signal indicative of a force applied on the sensor; and
a signal conditioning module with a communication element to communicate force sensor outputs to an external device.

22. The system of claim 21 further comprising a pressure sensor adapted to sense pressure within the bladder.

23. The system of claim 22 wherein the system communicates pressure sensor outputs from the pressure sensor to the external device.

24. The system of claim 23, wherein the external device is adapted to keep a time history of at least one of force and pressure.

25. The system of claim 21, wherein the force sensor is carried directly or indirectly by the inner surface of the brace.

26. The system of claim 21, wherein the force sensor is positioned to sense a force applied by the inflation bladder on the patient.

27. The system of claim 21, wherein the signal conditioning module is housed in a housing coupled directly or indirectly to an outer surface of the brace.

28. The system of claim 21, wherein the force sensor and the bladder are co-located between the patient and the brace.
